(19) United States
(12) Patent Application Publication
(10) Pub. No.: US 2001/0015203 A1
(43) Pub. Date: Aug. 23, 2001

(54) Breathing apparatus

(76) Inventor: Edward Cumming, Dorset (GB)

Correspondence Address: MARTINE PENILLA & KIM, LLP, 710 LAKEWAY DRIVE, SUITE 170, SUNNYVALE, CA, 94085, US

(21) Appl. No.: 09/730,628

(22) Filed: Dec. 5, 2000

(30) Foreign Application Priority Data
Dec. 7, 1999 (GB) 9928939.9

(51) Int. Cl. A62B 7/04; A61M 16/00; F16K 31/26

(52) U.S. Cl. 128/204.26

(57) ABSTRACT

A breathing apparatus is described, having a breathing circuit comprising a mouthpiece and one or more gas carrying conduits, a compressed gas source and a counterlung. The compressed gas source is in communication with the counterlung via the breathing circuit. The counterlung has an expansion assisting means and a contraction assisting means, and a control allows selective activation of the expansion assisting means and the contraction assisting means. The counterlung has primary and secondary chambers, and inflation of the secondary chamber causes inflation of the primary chamber. The expansion assisting means is a flow of compressed gas to inflate the secondary chamber.

### BACKGROUND TO THE INVENTION

The present invention relates to a breathing apparatus and particularly, but not exclusively, to breathing apparatus of the type commonly known as “rebreather” apparatus.

Conventional rebreather breathing apparatus comprise a compressed gas source, an expansible/collapsible reservoir (known as a counterlung), a mouth piece and breathing loop conduits, wherein the counterlung receives exhaled gas and inflates when a user breathes out into the mouth piece and deflates, feeding gas back to the mouthpiece when the user breathes in. Typically, the breathing loop includes a container containing a carbon dioxide absorbent material to prevent the build up of carbon dioxide which is exhaled by the user. Additionally, a rebreather apparatus typically includes a means for introducing oxygen into the breathing loop to replenish the oxygen which is consumed as the user breathes.

A rebreather apparatus provides the user with an extended period during which the apparatus can be used. This is because the oxygen in the breathing gas(es) may be circulated several times whereas a conventional “open-circuit” breathing apparatus exhausts the exhaled gases to the surrounding environment, which, of course, is wasteful since a significant proportion of the exhaled gases is oxygen.

There are three kinds of rebreather apparatus which are known, at least in the field of diving. These differ in the way in which they add gas to the breathing loop and control the concentration of oxygen in the gas fed to the breathing loop. The three different types of rebreather are known as: an oxygen rebreather, a semi-closed rebreather and a closed-circuit rebreather.

The oxygen rebreather includes a cylinder of pure oxygen to replace oxygen consumed by the user. In some cases this is applied to the breathing loop at a constant rate. However, since the metabolism of the user, such as a diver, is likely to vary during usage of the apparatus, it is necessary either to add too much oxygen which results in the wasteful venting of some of the oxygen from the loop during restful periods, or for the user to operate a manual by-pass valve to receive additional oxygen during periods of greater activity.

In most cases, however, the apparatus includes a means to attempt to match the supply oxygen to the breathing loop to the metabolic rate of the user. For example, as oxygen is used up, and carbon dioxide is removed by the scrubber, inhalation will eventually cause complete collapse of the counterlung. This can then be used to trigger an input of additional oxygen from the cylinder. However, there is a risk that the user may suffer from hypoxia (insufficient oxygen supply) before the trigger is activated.

More generally, with oxygen rebreathers there is always a risk of too much oxygen being supplied to the user which can give rise to oxygen toxicity. For this reason, oxygen rebreather apparatus cannot safely be used below six meters when diving.

The semi-closed rebreather includes a cylinder of mixed gas, the oxygen needed to replace that consumed by the user being mixed with helium and/or nitrogen. Again, there are available versions of this apparatus which supply gas at a constant rate and versions which attempt to match the rate of supply of the gas to the metabolic requirements of the user. Because of the build up of the non-consumed gas(es), there is a need for periodic venting of the breathing loop. The vented gas inevitably includes a proportion of oxygen. Overall, the efficiency of this type of apparatus in terms of the period of time until the gas supply is used up is relatively low (when compared to the other types of rebreather apparatus) because of this need for periodic venting.

The closed circuit rebreather apparatus generally includes two independent gas supplies, one of them being pure oxygen, the other, called the diluent, comprising a mixture of oxygen with either nitrogen and/or helium. Closed-circuit rebreather apparatus may include an electronic control system which includes an oxygen sensor to monitor the concentration of oxygen in the breathing gas and also a means for the automatic addition of oxygen when its partial pressure drops below a predetermined level. The main disadvantage of this type of rebreather apparatus is the risk of failure of the electronic system and the cost of the system.

A known arrangement for matching the volume of gas supplied to the respiration rate of the user in a semi-closed rebreather apparatus is to have two pairs of bellows, one inside the other. The outer pair of bellows serves as the counterlung and communicates with the inner pair of bellows via a first non-return valve. The inner pair of bellows serves as a pump for the venting or exhausting of gas through a second non-return valve. The outer pair of bellows communicates with the breathing loop and, as the diver inhales, both pairs of bellows collapse (the outer pair of bellows causing collapse of the inner pair of bellows) so that the portion of the gas which is present in the inner pair of bellows is automatically vented to the surrounding environment via the second non-return valve. The collapse of the counterlung also triggers replenishment of the gas from a mixed gas supply. When the user then exhales, both pairs of bellows expand (the outer pair of bellows causing expansion of the inner pair of bellows) and a portion of the exhaled air is transferred to the inner pair of bellows via the first non-return valve.

Although the system described immediately above is mechanically simple and reliable, it has the disadvantage that significant breathing resistance is encountered because the expansion and the collapse of the counterlung are both driven solely by the exhalation and inhalation of the user. To try to minimise this breathing resistance, large bore hoses are typically used in the breathing loop and the equipment is positioned on the user's body in such a way as to minimise this resistance.

### OBJECT OF THE INVENTION

An object of the present invention is to overcome, or at least ameliorate, some of the above-mentioned problems with known rebreather apparatus.

### SUMMARY OF THE INVENTION

According to a first aspect, the present invention provides a breathing apparatus including a breathing circuit including a mouthpiece and at least one gas carrying conduits, a compressed gas source and a counterlung, wherein the compressed gas source is in communication with the counterlung via the breathing circuit and wherein the counterlung includes an expansion assisting means and a contraction assisting means. The breathing apparatus preferably also includes a control to selectively activate the expansion assisting means or the contraction assisting means.

The expansion assisting means and the contraction assisting means of the counterlung assist the user to overcome the breathing resistance typically encountered in such systems and allows the user to breathe in a more normal way. Thus, the breathing effort required when using the breathing apparatus of the present invention is typically the same as that required for an open-circuit breathing apparatus, but the time for which the user can use the breathing apparatus of present invention is greatly increased. Accordingly, the present invention is likely to be far more appealing to the less experienced user than the presently known rebreather systems.

The counterlung preferably includes a primary chamber and a secondary chamber, wherein inflation of the secondary chamber causes inflation of the primary chamber. The secondary chamber preferably includes the expansion assisting means which more preferably comprises a flow of the compressed gas source to inflate the secondary chambers, which flow of compressed gas may be regulated by one or more valves.

The primary chamber preferably includes the contraction assisting means, which more preferably includes a spring, which is biassed towards contraction of the primary chamber.

In a preferred embodiment, the secondary chamber is formed within the primary chamber and more preferably, both the primary and secondary chambers are defined by a respective pair of bellows. However, in contrast to the known double bellows arrangement described above, the secondary chamber of this preferred embodiment is not in direct communication with the primary chamber, but instead communicates directly with the breathing circuit. In this preferred embodiment, the compressed gas source may be used selectively to inflate the secondary chamber which in turn causes inflation of the primary chamber and when the secondary chamber is deflated by the selective removal of the compressed gas, the contraction assisting means of the primary chamber, which is biassed towards contraction, urges the primary chamber to contract.

Advantageously, the breathing circuit includes a primary breathing circuit and a secondary breathing circuit, wherein the primary breathing circuit connects the primary gas chamber to the mouthpiece and the secondary breathing circuit connects the secondary chamber inter alia also to the mouthpiece.

The mouthpiece preferably includes a pressure operated mouthpiece switch such that when the user starts to inhale, the mouthpiece switch experiences a reduction in local or ambient pressure which results in the switch activating or operating a valve to allow gas from the primary gas chamber, via the primary breathing circuit, and gas from the secondary gas chamber, via the secondary breathing circuit, to enter a mouthpiece chamber, which is in communication with a mouthpiece outlet, thus enabling the gas to be breathed by the user.

Conversely, when the user starts to exhale, the mouthpiece switch experiences an increase in local pressure and activates or operates a second valve which allows compressed gas from the compressed gas source to enter the secondary gas chamber via the secondary breathing circuit. As the secondary gas chamber is inflated by the compressed gas, the exhaled gas from the user passes through the primary breathing circuit to the primary gas chamber. The exhalation by the user is thus assisted by the enforced expansion of the primary gas chamber by the secondary gas chamber.

Preferably, the mouthpiece is arranged such that only one of the first and second valves can be open at any one time. Thus, compressed gas is prevented from the entering the secondary breathing circuit during inhalation by the user.

In a further preferred embodiment, a non-return valve prevents exhaled gas entering the secondary breathing circuit.

The mouthpiece may include an exhaust valve to exhaust any excess gas that the user continues to exhale after the primary gas chamber is fully expanded.

Preferably the secondary breathing circuit includes a first conduit between the mouthpiece and the secondary gas chamber for carrying the compressed gas to the secondary gas chamber for inflation thereof and a second conduit for connecting the secondary gas chamber to the mouthpiece chamber for carrying gas from the secondary gas chamber to the mouthpiece. More preferably, the compressed gas source is additionally connected to the second conduit of the secondary breathing circuit via a top-up valve, the top-up valve being operable in the event that the primary chamber is completely collapsed. That is to say if the user wishes to inhale more gas than is contained in the primary and secondary gas chambers, then additional gas may be supplied to the mouthpiece chamber directly from the compressed gas source.

The breathing circuit of the present invention may also include a carbon dioxide scrubber which preferably comprises an absorbent material capable of absorbing carbon dioxide and removing it from the gas in the breathing circuit and a suitable container included within the circuit. In a preferred embodiment, the carbon dioxide scrubber is included in the primary breathing circuit.

As used herein, the term “gas” includes both a pure gas, eg. oxygen, and a mixture of gases.

### BRIEF DESCRIPTION OF THE DRAWINGS

Preferred embodiments of the present invention are described below, by way of example only, with reference to the accompanying drawings in which:

FIG. 1 is a schematic representation of one embodiment of a breathing apparatus according to the present invention; and

FIG. 2 is a schematic representation of a second embodiment of a breathing apparatus according to the present invention.

### DETAILED DESCRIPTION OF THE INVENTION

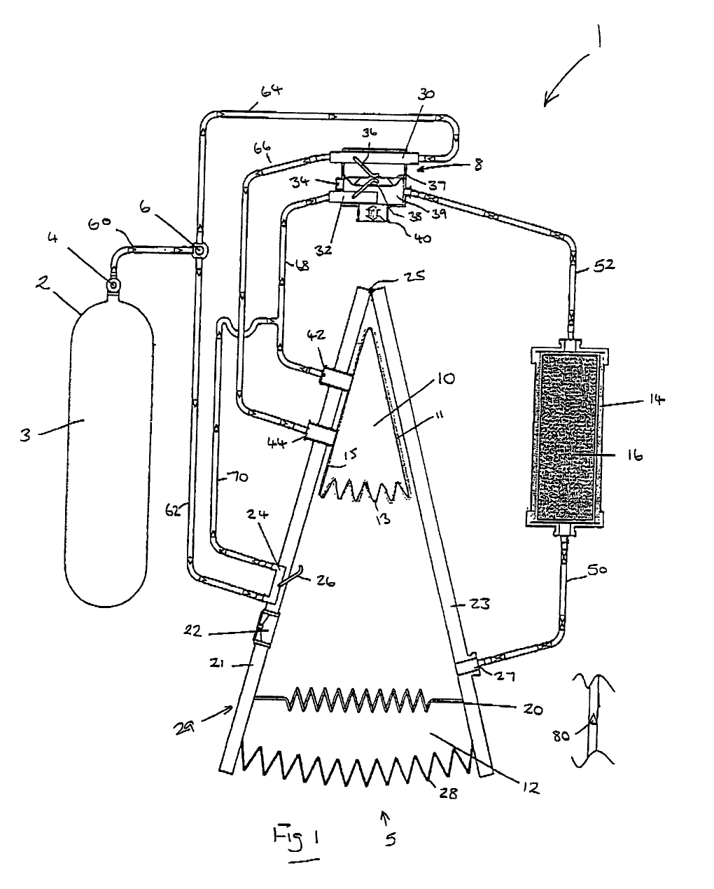

FIG. 1 shows a first embodiment of the present invention which comprises a cylinder 2 containing a mixed gas supply 3 consisting of a mixture of oxygen and nitrogen, a counterlung 5 and a mouthpiece 8.

The compressed gas cylinder 2 communicates with a pressure regulator 6 via conduit 60. The pressure regulator 6 communicates with the mouthpiece 8 via a conduit 64 and also with a gas replenishment valve 24 via a conduit 62.

The mouthpiece 8 includes a user interface 40 through which inhaled and exhaled gasses pass to and from a user's lungs. The mouthpiece 8 also includes what will be described as inhalation components and exhalation components. The inhalation components consist of an inhalation valve 32 controlled by a switch 38, and the exhalation components consist of an exhalation valve 30 controlled by a switch 36. Between the inhalation and exhalation components there is provided a diaphragm 37. The mouthpiece 8 also includes a primary breathing chamber 39 in communication with the user interface 40 and both a conduit 68 of a secondary breathing circuit and a conduit 52 of a primary breathing circuit.

When the diaphragm 37 is displaced towards the user interface 40, this causes the switch 38 to be displaced in the same direction which in turn opens the inhalation valve 32. Displacement of the diaphragm 37 back to its rest position causes movement of the switch 38 in the opposite direction which has the effect of closing the inhalation valve 32. The exhalation valve is opened in a similar way, except that in this case, the switch 36 is operated when the diaphragm 37 is urged away from the user interface 40. Again, the exhalation valve 30 is closed when the diaphragm 37 returns to its rest position (as shown in FIG. 1).

The counterlung 5 consists of a primary gas chamber 12 defined by a first pair of bellows 29 and a secondary gas chamber 10 defined by a second pair of bellows 11. As can be seen from FIG. 1, the secondary gas chamber 10 is located within the primary gas chamber 12.

The primary gas chamber 12 communicates with the primary breathing chamber 39 of the mouthpiece 8 via conduits 50 and 52 and a 2-way valve 27. Located between the conduits 50 and 52 is a carbon dioxide scrubber 14 which contains a carbon dioxide absorbent material 16.

The first pair of bellows 29 consists of two side walls 21, 23, joined at one end by a hinge 25 and joined at the other end by a flexible diaphragm 28. Located between the two walls 21, 23 is a helical spring 20 arranged to urge the two walls 21, 23 towards each other, ie. the spring is biassed towards the bellows adopting a closed configuration.

The second pair of bellows 11 includes a resilient side wall 15 and a flexible diaphragm 13. The second pair of bellows 11 communicates with the exhalation valve 30 of the mouthpiece 8 via a conduit 66 and a first non-return valve 44 and communicates with the inhalation valve 32 of the mouthpiece 8 via the conduit 68 and a second non-return valve 42. The conduit 68 also communicates with the outlet side of the gas replenishment valve 24 via a second gas replenishment conduit 70, the inlet side of the gas replenishment valve 24 being fed by the compressed gas 3 via a first gas replenishment conduit 62.

The breathing apparatus 1 also includes a first pressure release valve 22 which is located in the side wall 21 of the first pair of bellows 29 and a second pressure relief valve 34 which is located in the mouthpiece 8.

In use, starting with both primary gas chamber 12 and secondary gas chamber 10 being fully expanded, a user begins to inhale through the user interface 40. The negative pressure generated in the first chamber 39 causes the flexible diaphragm 37 to be drawn towards the user interface 40, which in turn opens the inhalation valve 32 via the switch 38. When the inlet valve 32 opens, the gas 3 in the secondary chamber 10 begins to pass into the primary breathing chamber 39 and the helical spring 20 urges the two side walls 21, 23 of the first pair of bellows to move towards each other, assisting the user to inhale the gas contained in both the primary gas chamber 12 via the primary breathing circuit consisting of the conduits 50 and 52 and the carbon dioxide scrubber 14, and the secondary gas chamber 10 via the secondary breathing circuit consisting of the conduit 68 and the inhalation valve 32.

When the user has completed his or her inhalation cycle. The primary breathing chamber 39 returns to normal ambient pressure which allows the flexible diaphragm 37 to return to its rest position, which in turn closes the inhalation valve 32.

Upon exhalation, the pressure in the primary breathing chamber 39 increases which urges the diaphragm 37 away from the user interface 40, which in turn opens the exhalation valve 30 via the switch 36. Once the exhalation valve 30 is open, the compressed gas 3 from the compressed gas cylinder 2 is allowed to pass into the secondary gas chamber 10 via the conduit 60, the pressure regulator 6, the conduit 64, the conduit 66 and the non-return valve 44. The compressed gas 2 entering the secondary chamber 10 causes the second set of bellows 11 to expand, which in turn causes the first pair of bellows 29 also to expand. The expansion of the first set of bellows 29 assists the user to exhale the waste gases from his of her lungs into the primary gas chamber 12 via the conduit 52, the carbon dioxide scrubber 14, the conduit 50 and the 2-way valve 27.

In the event that the user wishes to inhale more gas then is contained within the primary gas chamber 12 and the secondary gas chamber 10, this can be achieved via the gas replenishment valve 24. When the primary gas chamber 12 is nearly empty and the two side walls 21 and 23 of the first pair of bellows 29 are adjacent to one another, a switch 26 is operated by contact with the wall 23 which opens the gas replenishment valve 24. This allows the compressed gas 3 from the compressed gas cylinder 2 to flow into the secondary breathing circuit conduit 68 via the conduits 62 and 70. In other words, the user can draw the compressed gas 3 directly from the compressed gas cylinder 2 in the event that the first pair of bellows 29 completely collapses.

Additionally, if the user wishes to exhale more waste gas then there is capacity in the primary gas chamber 12 and the secondary gas chamber 10, then this excess waste gas can be exhausted either through the first pressure relief valve 22 or the second relief valve 34.

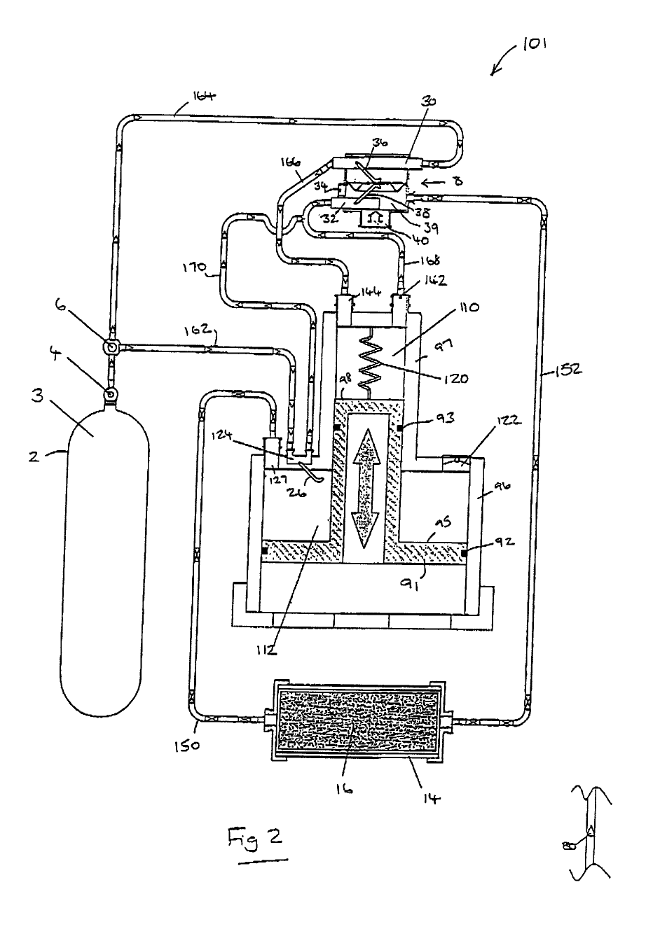

A second embodiment of the present invention is shown in FIG. 2. In this embodiment, the primary and secondary gas chambers are defined by a sliding piston 91. The piston 91 has a primary chamber defining surface 95 and a secondary chamber defining surface 98 and includes a first o-ring seal 92 and a second o-ring seal 93. The primary gas chamber 112 is defined by the primary gas chamber defining surface 95 of the piston 91 and a cylinder wall 96. The secondary gas chamber is defined by the secondary gas chamber defining surface 98 of the piston 91 and a cylinder wall 97.

The arrangement of the primary and the secondary breathing circuit conduits is similar to that described with respect to the embodiment shown in FIG. 1, with the corresponding conduits shown in FIG. 2 having the prefix “1” before their reference numerals. The components which are common to both embodiments have the same reference numerals in both Figures and need not be described again.

The breathing apparatus works in a similar way as that described above with regard to embodiment shown in FIG. 1. During the inhalation of the user, the inhalation valve 32 is opened which allows the helical compression spring 120 to urge the piston 91 in an upward direction (as shown in FIG. 2) to urge the gasses in the primary gas chamber 112 and the secondary gas chamber 110 into the mouthpiece 8 via the primary and secondary circuits. During the exhalation of the user, compressed gas 3 from the cylinder 2 is allowed to pass into the secondary gas chamber 110, which overcomes the compression force of the spring 120 and urges the piston 91 in a downward direction (as shown in FIG. 2) which assists the user to exhale his or her waste gasses into the primary gas chamber 112 via the carbon dioxide scrubber 14.

These preferred embodiments have been described by way of an example and it will be apparent to those skilled in the art that many alterations can be made that are still within the scope of the invention.

1. A breathing apparatus including a breathing circuit, the breathing circuit including:
a mouthpiece;
at least one gas carrying conduit;
a compressed gas source; and
a counterlung,
wherein the compressed gas source is in communication with the counterlung via the breathing circuit and wherein the counterlung includes an expansion assisting means and a contraction assisting means.

2. A breathing apparatus according to claim 1 further including a control to selectively activate one of the expansion assisting means and the contraction assisting means.

3. A breathing apparatus according to claim 1 wherein the counterlung includes:
a primary chamber, and
a secondary chamber,
wherein inflation of the secondary chamber causes inflation of the primary chamber.

4. A breathing apparatus according to claim 3 wherein the expansion assisting means are associated with the secondary chamber.

5. A breathing apparatus according to claim 4 wherein the expansion assisting means comprises a flow of the compressed gas source.

6. A breathing apparatus according to claim 3 wherein the contraction assisting means is associated with the primary chamber.

7. A breathing apparatus according to claim 6 wherein the contraction assisting means is a spring which is biased towards contraction of the primary chamber.

8. A breathing apparatus according to claim 1 wherein the secondary chamber is formed within the primary chamber.

9. A breathing apparatus according to claim 8 wherein the secondary chamber communicates directly with the breathing circuit and does not communicate directly with the primary chamber.

10. A breathing apparatus according to claim 3 wherein the breathing circuit includes:
a primary breathing circuit, and
a secondary breathing circuit,
wherein the primary breathing circuit connects the primary gas chamber to the mouthpiece and the secondary breathing circuit connects the secondary chamber to the mouthpiece.

11. A breathing apparatus according to claim 10 wherein the mouthpiece includes:
a pressure operated mouthpiece switch;
a first valve;
a mouthpiece chamber; and
a mouthpiece outlet,
the switch being operable to respond to a reduction in pressure to operate the valve to allow gas from the primary gas chamber, via the primary breathing circuit, and gas from the secondary gas chamber, via the secondary breathing circuit, to enter the mouthpiece chamber, the mouthpiece chamber being in communication with the mouthpiece outlet.

12. A breathing apparatus according to claim 10 wherein the mouthpiece includes:
a pressure operated mouthpiece switch;
a first valve;
a second valve;
a mouthpiece chamber; and
a mouthpiece outlet,
the switch being operable to respond to an increase in pressure to operate the second valve to allow compressed gas from the compressed gas source to enter the secondary gas chamber via the secondary breathing circuit.

13. A breathing apparatus according to claim 12 wherein only one of the first and second valves can be opened at any one time.

14. A breathing apparatus according to claim 10 wherein the apparatus further includes a non-return valve to prevent exhaled gas from entering the secondary breathing circuit.

15. A breathing apparatus according to claim 3 wherein the mouthpiece includes an exhaust valve to exhaust, in use, any excess gas that a user continues to exhale after the primary gas chamber is fully expanded.

16. A breathing apparatus according to claim 10 wherein the secondly breathing circuit includes:
a first conduit for carrying the compressed gas to the secondary gas chamber for inflation thereof; and
a second conduit for connecting the secondary gas chamber to the mouthpiece chamber for carrying gas from the secondary gas chamber to the mouthpiece.

17. A breathing apparatus according to claim 16, further including a top-up valve, the compressed gas source being connected to the secondary conduit of the secondary breathing circuit via the top-up valve, the top-up valve being operable in the event that the primary chamber is completely collapsed.

18. A breathing apparatus according to claim 1, further including a carbon dioxide scrubber.

19. A breathing apparatus according to claim 10, further including a carbon dioxide scrubber, wherein the carbon dioxide scrubber is included in the primary breathing circuit.

20. A breathing apparatus including a breathing circuit, the breathing circuit including:
a mouthpiece;
at least one gas carrying conduit;
a compressed gas source;
a counterlung,
wherein the compressed gas source is in communication with the counterlung via the breathing circuit and wherein the counterlung includes:
a primary chamber;
a secondary chamber;
an expansion assisting means associated with the secondary chamber;
a contraction assisting means which is a spring which is biased towards contraction of the primary chamber; wherein the secondary chamber is formed within the primary chamber and wherein inflation of the secondary chamber causes inflation of the primary chamber,
the breathing circuit further including:
a primary breathing circuit;
a secondary breathing circuit; and
a carbon dioxide scrubber,
wherein the primary breathing circuit connects the primary gas chamber to the mouthpiece and the secondary breathing circuit connects the secondary chamber to the mouthpiece, and wherein the carbon dioxide scrubber is included in the primary breathing circuit.
